(19) United States
(12) Patent Application Publication
(10) Pub. No.: US 2016/0333516 A1
(43) Pub. Date: Nov. 17, 2016

(54) Energy Infusion Process

(71) Applicant: Stephen Taylor, Albuquerque, NM (US)

(72) Inventor: Stephen Taylor, Albuquerque, NM (US)

(21) Appl. No.: 14/711,207

(22) Filed: May 13, 2015

(51) Int. Cl. D06M 10/00; A61K 8/97; A61K 8/31; A61K 8/55; A61K 8/63; B01J 19/12; A61K 8/68; A61K 8/92; A61K 8/49; A61K 8/24; A61K 8/19; A61Q 3/00; A61K 8/67

(52) U.S. Cl. CPC D06M 10/001; A61Q 3/00; A61K 8/97; A61K 8/31; A61K 8/553; A61K 8/63; A61K 8/67; A61K 8/68; A61K 8/922; A61K 8/4986; A61K 8/24; A61K 8/19; B01J 19/122; A61K 2800/81

(57) ABSTRACT

Methods to treat and produce articles of manufacture that are activated with an electromagnetic and electrical charge to produce activated articles of manufacture and formulations are provided garments. One aspect of the invention is directed toward activated garments and apparel. Our results described herein showed that the fabric sample treated with the activation process according to the invention released more electrons than the control sample. The treated fabric samples released electrons with a lower average energy, making the water of the treated fabric sample more prone to release electrons. Another aspect of the invention is directed toward providing methods and activated formulations for treating various aliments. One formulation described is an activated topical formulation for improving the strength and vitality of nails.

### CROSS-REFERENCE TO RELATED APPLICATIONS

This application takes priority from U.S. Ser. No. 61/992,668 by Stephen Kit Taylor entitled ‘Formulations and methods for improving nail health vitality’ filed on May 13, 2014, the contents of which are incorporated herein in their entirety.

### FIELD

The present disclosure relates to a process for infusing electromagnetic, magnetic and/or electrical energy to formulations and articles of manufacture.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Electromagnetic fields have been used to treat or suppress various medical disorders and maladies, and are typically applied through electrodes which are directly in contact internally or externally to a target area of a patients body. It has recognized that electronically stimulating specific points in a patient's body are beneficial in treating bone repair and regrowth, dermatological applications, treatment of burns, sleep disorders, stress, TMJ problems, bruxism, headaches, and in general for injury, repair, and muscular relaxation in the body. To the inventors knowledge, charge and electromagnetic field generation devices have, while used on subjects directly, not been used indirectly for ingestible foods or medicines, formulations, devices, materials, and articles of manufacture that are indirectly associated with a subject or patient.

There are a large number and variety of devices generally designed to assist individuals though the application of an electrical current to a body part. A number of efforts have been made in the past to utilize an energy source in the treatment of pain, tension, and various medical and psychiatric conditions. Many of these methodologies involve the use of radio frequency, electromagnetic energy, resonant acoustic energy, or therapeutic resonant frequencies. Electromagnetic fields are commonly used to treat or suppress various medical disorders and maladies, and are typically applied through electrode which are directly in contact internally or externally to a target area.

To the inventors knowledge, however, the application of electromagnetic and magnetic fields and charged energy has been thus far limited direct application to the body of a subject. Accordingly, one area of need and neglect, however, has been to treat articles of manufacture, compositions and formulations for topical, oral, or internal administration with an electromagnetic or electrical charge to produce electromagnetically and/or electrically activated or charged article or formulations. Numerous embodiments are provided herein that satisfy the unmet need of applying electromagnetic and electric charge energy to articles of manufacture, formulations, and a wide range of targets for application of the activation methods described herein other than direct application of an electromagnetic field or current directly to a subject.

One exemplary area of particular need is for an electromagnetically and/or electrically activated topical formulation for administration to finger and toe nails and associated tissues. A number of nail strengthening products are currently on the market, but the majority of them are directed toward applying a hardening surface agent on the surface of the nail. A problem with these products is that the formulation and methods used are to apply the formulations to the surface of nails much like a paint that stays on the surface without getting absorbed. What is needed in this instance is a topical formulation that can be applied to finger or toe nails and have active ingredients which effectively promote the strength, health, and vitality of nails and also combat fungal infections and other aliments commonly suffered in nails.

The invention also satisfies this particular need. To meet this need, particular embodiments of the invention are directed to novel formulations provided by subjecting the nail formulations with an electromagnetic energy stimulus to produce a charge activated nail formulation. The activated nail formulation is not only activated with electromagnetic energy, it is formulated to penetrated into the growth centers and nail bed, thus allowing for infusion and utilization of the activated formulation and necessary nutrients and minerals to help the body to grow and create healthy nails on its own. This gives the body an advantage for repairing and regrowing the weakened supportive tissue, nails and nail bed, thereby producing a healthy, strong finger or toe nail. The potential use of electromagnetic fields for the treatment of maladies in human nails and toes is a novel concept has never been considered or implemented effectively to date to the best of the inventors knowledge. Accordingly, this embodiment and others described herein satisfy a need for improved topical skin and nail treatment formulations.

Another area of neglect, for example, has been to treat articles of apparel, clothing, and the like that are tightly associated with a subject. To the inventors knowledge, there is an unmet need for articles of manufacture such as apparel and clothing that are activated with an electromagnetic and electrical charge. Another aspect of the invention is directed toward activated garments and apparel. Our results described herein showed that the fabric sample treated with the activation process according to the invention released more electrons than the control sample. The treated fabric samples released electrons with a lower average energy, making the water of the treated fabric sample more prone to release electrons. Activated garments were made from the methods described herein, and the users that wore the activated clothing items showed an increase in strength and motion. Accordingly, this embodiment serves the need of providing energy stimulating, strength enhancing, and muscle soreness and injury relief by virtue of the beneficial affects of the novel methods used herein to produce electromagnetic field and charge and activated articles and formulations.

These embodiments and others directed to other ingestible foods, formulations, devices, materials, and articles of manufacture that have been activated by the methods described herein are provided.

### BRIEF SUMMARY

The inventions described and claimed herein have many attributes and embodiments including, but not limited to, those set forth or described or referenced in this

Brief Summary. The inventions described and claimed herein are not limited to, or by, the features or embodiments identified in this Summary, which is included for purposes of illustration only and not restriction.

There are several aspects to the various inventions provided herein because the inventions provided herein are useful for a wide range of applications. The inventions provided herein are typically related to methods to produce articles of manufacture, devices, and formulations with an enhanced electromagnetic energy, charge, and/or energy level.

Accordingly, in one embodiment of the invention a method of producing an article of manufacture is provided that comprises the steps of selecting an article of manufacture, providing a electromagnetic energy frequency generator, placing the selected article of manufacture in direct or indirect contact with the frequency generator, selecting electromagnetic energy output frequency and voltage levels and applying electromagnetic energy to the article of manufacture for a predetermined amount of time. Such a process according to the invention produces an article of manufacture that retains that electromagnetic energy and/or electrical charge.

Activated apparel and clothing items are examples of activated articles of manufacture provided herein.

In other embodiments, electromagnetic and/or charge activated formulations are provided. Formulations suitable for ingestion are provided, as well as topical formulations. One preferred topical formulation provided herein is for improving the strength, health and vitality of nails and related tissues. The formulation is treated to infuse it with electromagnetic radiation and/or a charge, preferably before it is applied or administered to a subject, and the formulation comprises at least ten of the following nutraceuticals, fatty acids, proteins, and minerals: squalane; rose hip (Rosa Rubinosa); pomegranate (Rumica Granatum); chia (Salvia Hispanica) seed oil; soybean (Glycinemax, non GMO); lecithin; a cholesterol selected from i) cholesterol oleyl carbonate, ii) cholesterol annote, and iii) cholesterol chloride; astaxanthin; tocotriends; lycopene; alpha d tocopherol; thioctic acid; lutin; beta carotene; ceramide 3; phytosteriods; gamma oryzanol; vitamin D3; manganese; phosphorous; a calcium selected from i) calcium orotate and ii) calcium piccolinate; magnesium; and one or more carrier, excipient, or the like suitable for topical administration.

Another aspect of the invention is to provide systems and kits. One such embodiment, for example, is directed to a kit for improving the strength, health, vitality, or appearance of nails, the kit comprising i) a formulation provided herein, and ii) instructions for methods of use of said kit according to one of the methods provided herein.

### DETAILED DESCRIPTION

Various aspects of the invention will now be described with reference to the following experimental section which will be understood to be provided by way of illustration only and not to constitute a limitation on the scope of the invention.

In one aspect, devices are provided which are used to transmit electromagnetic energy to provided electromagnetic and/or charge energy activated articles of manufacture, devices, and formulations herein. Preferred articles and formulations are capable of conducting and absorbing the electromagnetic energy and sometimes then acting as a conducting medium. An exemplary electromagnetic and/or charge activated article is produced by i) selecting an article of manufacture or formulation; ii) providing a electromagnetic energy frequency generator; and iii) placing the selected article of manufacture in direct or indirect contact with the frequency generator. Next, (step iv) electromagnetic energy output frequency and voltage levels are selected that are suitable for the article of manufacture selected in step i). Electromagnetic energy is applied to the article of manufacture for a predetermined amount of time to produce an article of manufacture that retains that electromagnetic energy and/or electrical charge. For the purpose herein, an ‘electromagnetic energy frequency generator’ (otherwise known as a ‘frequency generator’ or ‘electromagnetic frequency generator’) may include anything that generates any kind of electromagnetic energy, magnetic energy, electrical energy, audible and inaudible sound frequencies, other electromagnetic radiation (e.g. IR, UV, light waves, etc.) charge or the like. A electromagnetic frequency generator may thus include anything from a dedicated set of electronic equipment, to anything that generates a desired current (or micro-current), electrical energy, charge, and/or sound or magnetic field energy.

In one embodiment, electromagnetic radiation is applied by a frequency generator that is placed in direct physical contact with the selected materials. In another embodiment, electromagnetic radiation is applied by a frequency generator that is placed in indirect physical contact with the selected material or article of manufacture. In another embodiment, the selected article of manufacture is placed in direct contact with a fluid (e.g. a bath) in direct contact with a frequency generator. An exemplary fluid is a bath of water which is at least partially covering the selected material or article of manufacture. In one preferred embodiment, the frequency generator is comprised of electrodes that are placed in direct contact with a bath of fluid that is primarily water.

The electromagnetic radiation that is applied is typically of a pre selected frequency or wavelength, duration, and intensity. In addition to sound, a fluctuating electromagnetic (EM) field generated by a radio (RF) oscillator, an infrared (IR) light generating source (in the infrared spectral range), or other electromagnetic radiation generation sources are used to infuse the selected article in alternative embodiments Implementation of the charge typically originates in a power source, an oscillator, (or other frequency-generating device), electrodes, a control device, an optimal timer, and a host appliance and generates an electromagnetic charge. When this frequency is between 2,000 HZ (2 KHz) and 15,000 HZ (15 KHz), the electromagnetic frequency held by the formulation maintains the electromagnetic charge, and generates the frequency output on its own.

Articles of Manufacture.

Activated electromagnetic and charge infused articles of manufacture that are treated by the methods and provided herein include medical and oral devices, dental devices, cosmetics, skin care, lotions and creams, and related articles, as well as consumable and ingestible products, foods, candies, and other consumables.

A preferred non-limiting embodiment of an article of manufacture according to the invention is directed to fabrics, apparel and articles of clothing manufacture made according to the methods described herein. Non-limiting examples of fabrics used include those from a variety of natural and man-made materials, fibers, and fabrics including for example polyester, hemp, and Spandex™.

Other embodiments are directed to various apparel gear that has been activated according to the methods provided herein, including for example short sleeve shirts (crew cut, aV neck, long neck, etc.), tank top shirts, vest, workout gear, compression sports wear (e.g.

workout compression shorts), padded bicycle shorts, elbow sleeves, knee braces (with and without copper and magnets), swim wear, wet suits, mouth guards, chin straps, lower and upper back braces, protective ankle braces (with and without pliable inserts), support hose and socks, helmet and helmet pads (e.g. hockey, bicycle, and football inner helmet pads).

Formulations and Compositions.

In still other embodiments, the method is applied to formulations, chemicals, and living organisms. In preferred embodiments of the invention, formulations provided herein comprise of a mixture of vitamins, minerals, cellular regeneration nutrients and herbal extracts that have anabolic and protein rebuilding properties. A human cell encompasses any kind of cell within the body, and such cells typically need an ongoing supply of oxygen and nutrients on a regular basis. Most cells need a replenishment of oxygen and nutrients every 15 to 30 seconds. The vascular system is largely responsible for delivering the necessary nutrients to the tissues and cells. It is now common and accepted scientific knowledge that our bodies can absorb nutrients and minerals not only through blood, but through our skin and connective tissue. This adsorption process through skin and related tissues is enhanced by the formulations and infusion process provided herein.

The formulations provided herein preferably utilize a pomegranate oil base. This oil is very rich in anti oxidants and omega 3 fatty acids. This aids the body in healing diseased or damaged tissue and in creating healthy neuro-transmitters. Cellular absorption is the key to good strong, healthy human tissue. Disease process with few exceptions (certain cancers, etc.) are caused, facilitated or exacerbated primarily to a weakened immune process or system. When healthy vibrant cells are present it is very rare to find an overload of bacteria, viruses, spores or fungi.

As discussed above, using methods of the invention, the electromagnetic frequency held by an activated formulation maintains the electromagnetic charge and generates the frequency output on its own. In the case of the nail formulations provided herein, this then enables the receipt ingredients to pass though the skin and connective tissue to be readily available for absorption into the cells. While not wishing to be bound to any theory, this new and novel technique is thought to work in one manner to emulsify the active ingredients in the formulation and makes them more readily absorbable. This electromagnetic energy infusion is also believed to increase the surface area of the affected cells to allow for greater and easier cellular absorption.

A primary objective of certain embodiments of the invention is to infuse the necessary nutrients and minerals directly into the nail, nail bed and cuticle to assist the body in building strong and healthy cells. This strengthens the finger and toe nails, supporting them in growing or regrowing into strong healthy structures. In certain embodiments provided herein, this is accomplished by treating the nail composition with a selected frequency of electromagnetic radiation to substantially enhance the absorption of the formulation and thus its effectiveness.

Using a non toxic, pure, natural nail hardening formulation deep into the nails (finger or toe), nail bed and supportive tissue. Using the formulations and methods provided herein, this creates greater absorption and excites the neuro-receptors in the finger and toe areas, thereby sending afferent neural impulses to the central nervous system, further potentiating neural support and responses.

Some formulations of the invention are directed to topical compositions for improving the strength, health and vitality of nails and related tissues. In typical embodiments of these compositions, the formulation comprises at least ten of the following nutraceuticals, fatty acids, proteins, and minerals: squalane; rose hip (Rosa Rubinosa); pomegranate (Rumica Granatum); chia (Salvia Hispanica) seed oil; soybean (Glycinemax, non GMO); lecithin; a cholesterol selected from i) cholesterol oleyl carbonate, ii) cholesterol annote, and iii) cholesterol chloride; astaxanthin; tocotriends; lycopene; alpha d tocopherol; thioctic acid; lutin; beta carotene; ceramide 3; phytosteriods; gamma oryzanol; vitamin D3; manganese; phosphorous; a calcium selected from i) calcium orotate and ii) calcium piccolinate;

magnesium; and one or more carrier, excipient, or the like suitable for topical administration. In one particular embodiment, a topical formulation for improving the strength, health and vitality of nails and related tissues is provided that comprises at least ten of the following nutraceuticals, fatty acids, proteins, and minerals: squalane in an amount of between about 5 to 7% by weight; rose hip (Rosa Rubinosa) in an amount of between about 8 to 10% by weight; pomegranate (Rumica Granatum) in an amount of between about 7 to 10% by weight; chia (Salvia Hispanica) seed oil in an amount of between about 5 to 8% by weight; soybean (Glycinemax, non GMO) in an amount of between about 5 to 8% by weight; lecithin in an amount of between about 3 to 5% by weight; a cholesterol selected from i) cholesterol oleyl carbonate, ii) cholesterol annote, and iii) cholesterol chloride in an amount of between about 8 to 9% by weight; astaxanthin in an amount of between about 3 to 5% by weight; tocotriends in an amount of between about 3 to 5% by weight; lycopene in an amount of between about 3 to 5% by weight; alpha d tocopherol in an amount of between about 2 to 3% by weight; thioctic acid in an amount of between about 3 to 5% by weight; lutin and beta carotene in an amount of between about 3 to 5% by weight in combination; ceramide 3 in an amount of between about 2 to 3% by weight; phytosteriods in an amount of between about 3 to 5% by weight; gamma oryzanol in an amount of about 2% by weight; vitamin D3 in an amount of between about 3 to 5% by weight; manganese in an amount of between about 2 to 3% by weight; phosphorous in an amount of between about 1 to 3% by weight; a calcium selected from i) calcium orotate and ii) calcium piccolinate in an amount of about 3% by weight; magnesium in an amount of between about 2 to 3% by weight; and one or more carrier, excipient, or the like suitable for topical administration. In alternative embodiments of the formulations provided above, instead of only ten of the compounds listed, the formulations may comprise at least 12, at least 15, at least 18, at least 20, or all of the listed nutraceuticals, fatty acids, proteins, and minerals.

In another aspect, the formulations provided herein are optionally activated by treating the formulation by with the infusion of electromagnetic radiation of a pre selected frequency or wavelength, duration, and intensity. The electromagnetic radiation utilized for infusion into formulations may constitute a wide range of frequencies such as sound waves within or out of the audible spectrum in humans, or as another example by electromagnetic energy in the infrared spectral range an infrared (IR) light. In these embodiments, treatment with sound waves increases the effectiveness of the formulation(s) by enhancing the absorption of formulation when it is applied to the nails and associated tissues of a subject (e.g. human, or certain animals with tissues related to human nail tissue). In some embodiments, the infusion of electromagnetic radiation is achieved by a direct contact of a formulation provided herein with an AC voltage source having a selected frequency. In some embodiments, the infusion of electromagnetic radiation is achieved by infusing an electromagnetic energy developed by a non-contact fluctuating electromagnetic field generator having a selected frequency. In some embodiments, the infusion of electromagnetic radiation is achieved by infusing an electromagnetic energy developed by a fluctuating electromagnetic (EM) field generated by a radio (RF) oscillator.

The compositions provided herein are typically applied to nails and related tissues including the finger nails, toe nails, cuticles, nail bed, and growth centers of nails of a human. However, there are also veterinary applications for the formulations provided herein for the treatment of hoofs and tissues related to nails in mammals such as the hoofs of race horses or livestock. The formulations provided herein are useful for various methods of treating infections (e.g. fungal infections) and improving the strength, health, vitality, or appearance of nails.

Accordingly, another aspect of the invention is directed towards methods of utilizing the formulations provided herein. In one embodiment, a method of improving the strength, health, vitality, or appearance of nails, the method is provided that follows the steps of i) evaluating the finger nails or toe nail problems of a particular subject, ii) selecting a formulation provided herein, iii) selecting a treatment regimen for that particular individual based upon their need, iv) applying said formulation to one or more of the finger nails, toe nails, cuticles, nail bed, and growth centers of nails of a human according to the selected treatment regimen, and v) monitoring the success of the treatment based upon the needs of the patient and continuing said treatment until a selected outcome is achieved. Other embodiments are directed to methods for treating a fungal infection in a nail following the steps of i) evaluating the extent and severity of a fungal infection in the finger nails or toe nails of a particular subject, ii) selecting a formulation provided herein, iii) selecting a treatment regimen for that particular individual based upon their need, iv) applying said formulation to one or more of the finger nails, toe nails, cuticles, nail bed, and growth centers of nails of a human according to the selected treatment regimen, and v) monitoring the success of the fungal infection treatment until a selected outcome is achieved.

Another aspect of the invention is the use of methods of tactile stimulation to enhance the adsorption and effectiveness of certain topical formulations provided herein. Another method of treating a fungal infection in a nail provide herein follows the steps of i) evaluating the extent and severity of a fungal infection in the finger nails or toe nails of a particular subject, ii) selecting a formulation provided herein, iii) selecting a treatment regimen for that particular individual based upon their need, iv) applying said formulation to one or more of the finger nails, toe nails, cuticles, nail bed, and growth centers of nails of a human according to the selected treatment regimen, said application involving a tactile stimulation of any one of finger nails, toe nails, cuticles, nail bed, and growth centers of nails sufficient to trigger a neuroreceptor response to facilitate a desired positive physiological change beneficial for the treatment of said fungal infection, and v) monitoring the success of the fungal infection treatment until a selected outcome is achieved. A preferred embodiment of the above described invention involves the administration of a formulation provided herein to one of finger nails, toe nails, and affected surrounding tissues including cuticles, nail bed, and growth centers of the nails where the step of rubbing the formulation into the affected areas is performed until the formulation is fully absorbed into the tissues to which it is applied.

### Neuro-Physiological Effects.

Our nervous system is directly influenced by impulses coming from our neuro-sensory receptors. Once a receptor is brought to threshold (a frequency generated process), it fires the corresponding nerve. Mechanical receptors are mechanically stimulated and are responsible for our equilibrium, hearing, blood pressure, digestion, and all muscular contractions and relaxation. These receptors may be free nerve endings in our epidermis and the underlying dermis of our skin. Also involved are Pacinian pressure corpuscles, which are located though out all of the tissues of our bodies, as well as Merkles Disc, Meissner's Corpuscles, Ruffini's End Organ, muscle spindles and golgi tendons, which are all are physiologically involved in our fingers and toes. Certain methods provided herein involve rubbing the formulation into the nails and nail beds and cuticles of our finger and toes, which stimulates a large variety of receptors thereby stimulating the nervous system and assessing the brain of the need and change necessary for strengthening nails the related nail structures.

To understand Frequency Dr. Arthur C Guyton, M.D. writes in his nationally known textbook “Basic Neuroscience”. “Basically, any factor that causes Sodium Ions to begin to diffuse inward through the neural membrane sufficient numbers will set off the autonomic, regenerating opening of the sodium channels. This can result from simple mechanical disturbance of the membrane, chemical, chemical effects on the membrane, or passage of electricity through the membrane. All of these are used at different point in the body to elicit nerve or muscle action potentials: mechanical pressure to excite sensory nerve endings in the skin, chemical neurotransmitters to transmit signals from one neuron to the next in the brain, and electrical currant to transmit signals between muscles cells to include skeletal, cardiac, and smooth muscle.”

The present invention discloses the use of a electromagnetic, frequency/static charge that can be incorporated into the formulations provided herein and effects and stimulates the neuroreceptors therby increasing the central integrative state of the neuron and this directly affects the Central Nervous System. By raising the central integrative state of the neuron, it requires a lower stimulus to bring the nerve to threshold and therefore lowers the stimulus to fire the neuron. Through this frequency stimulation, neuro-plasticity occurs and sets up a re-excitation process necessary for rhythmicity (see Guyton above). In effect this stimulates higher brain centers.

The body's cellular function is dependent upon the frequencies of energy that it is producing or responding to. By incorporating specific electrical, light sand sound frequencies into the formulations of the invention, the use of it increases the overall healing ability of the damaged nails and nail structures.

The infused serum carries a low-amperage frequency in the range of 2,000 Hz (2 KHz) to 15,00 Hz (15 KHz) into the Nails and supportive Nail structures

The following Examples are included for illustration and not limitation.

### EXAMPLE 1

This example is directed to the preparation of various formulations of the invention:

Formulation 1:

A topical formulation comprising 5% by weight squalane; 8% by weight rose hip (Rosa Rubinosa); 7% by weight pomegranate (Rumica Granatum); 5% by weight chia (Salvia Hispanica) seed oil; 5% by weight soybean (Glycinemax, non GMO); 3% by weight lecithin; 8% by weight cholesterol oleyl carbonate (cholesterol annote or cholesterol chloride can be substituted); 3% by weight astaxanthin; 3% by weight tocotriends; 3% by weight lycopene; 2% by weight alpha d tocopherol; 3% by weight thioctic acid; 1% by weight lutin; 1% by weight beta carotene; 2% by weight ceramide 3; 3% by weight phytosteriods; 2% by weight gamma oryzanol; 3% by weight vitamin D3; 2% by weight manganese; 1% by weight phosphorous; 2% by weight calcium orotate (calcium piccolinate can be substituted); 2% by weight magnesium; and a balance of palm oil as a carrier (suitable oil carrier, excipient, or the like suitable for topical administration may be substituted).

Formulation 2:

A topical formulation comprising 7% by weight squalane; 10% by weight rose hip (Rosa Rubinosa); 10% by weight pomegranate (Rumica Granatum); 8% by weight chia (Salvia Hispanica) seed oil; 8% by weight soybean (Glycinemax, non GMO); 5% by weight lecithin; 10% by weight cholesterol oleyl carbonate (cholesterol annote or cholesterol chloride can be substituted); 5% by weight astaxanthin; 5% by weight tocotriends; 5% by weight lycopene; 3% by weight alpha d tocopherol; 5% by weight thioctic acid; 2.5% by weight lutin; 2.5% by weight beta carotene; 3% by weight ceramide 3; 5% by weight phytosteriods; 2% by weight gamma oryzanol; 5% by weight vitamin D3; 5% by weight manganese; 3% by weight phosphorous; 3% by weight calcium orotate (calcium piccolinate can be substituted); 2% by weight magnesium; and a balance of palm oil as a carrier (suitable oil carrier, excipient, or the like suitable for topical administration may be substituted).

Formulation 3:

A topical formulation comprising 7% by weight squalane; 10% by weight rose hip (Rosa Rubinosa); 10% by weight pomegranate (Rumica Granatum); 8% by weight chia (Salvia Hispanica) seed oil; 8% by weight soybean (Glycinemax, non GMO); 5% by weight lecithin; and 10% by weight cholesterol oleyl carbonate (cholesterol annote or cholesterol chloride can be substituted); and a balance of palm oil as a carrier (suitable oil carrier, excipient, or the like suitable for topical administration may be substituted).

Formulation 4:

A topical formulation comprising 10% by weight squalane; 15% by weight rose hip (Rosa Rubinosa); 15% by weight pomegranate (Rumica Granatum); 12% by weight chia (Salvia Hispanica) seed oil; 12% by weight soybean (Glycinemax, non GMO); 7% by weight lecithin; and 15% by weight cholesterol oleyl carbonate (cholesterol annote or cholesterol chloride can be substituted); and a balance of palm oil as a carrier (suitable oil carrier, excipient, or the like suitable for topical administration may be substituted).

Formulation 5:

A topical formulation comprising 7% by weight squalane; 10% by weight rose hip (Rosa Rubinosa); 10% by weight pomegranate (Rumica Granatum); 8% by weight chia (Salvia Hispanica) seed oil; 8% by weight soybean (Glycinemax, non GMO); 5% by weight lecithin; 10% by weight cholesterol oleyl carbonate (cholesterol annote or cholesterol chloride can be substituted); 5% by weight astaxanthin; 5% by weight tocotriends; 5% by weight lycopene; and 3% by weight alpha d tocopherol; and a balance of palm oil as a carrier (suitable oil carrier, excipient, or the like suitable for topical administration may be substituted).

Formulation 6:

A topical formulation comprising 7% by weight squalane; 10% by weight rose hip (Rosa Rubinosa); 10% by weight pomegranate (Rumica Granatum); 8% by weight chia (Salvia Hispanica) seed oil; 8% by weight soybean (Glycinemax, non GMO); 5% by weight lecithin; 10% by weight cholesterol oleyl carbonate (cholesterol annote or cholesterol chloride can be substituted); 5% by weight astaxanthin; 5% by weight tocotriends; 5% by weight lycopene; 3% by weight alpha d tocopherol; 5% by weight thioctic acid; and 2.5% by weight lutin; and a balance of palm oil as a carrier (suitable oil carrier, excipient, or the like suitable for topical administration may be substituted).

Formulation 7:

A topical formulation comprising 5% by weight squalane; 8% by weight rose hip (Rosa Rubinosa); 7% by weight pomegranate (Rumica Granatum); 5% by weight chia (Salvia Hispanica) seed oil; 5% by weight soybean (Glycinemax, non GMO); 3% by weight lecithin; and 8% by weight cholesterol oleyl carbonate (cholesterol annote or cholesterol chloride can be substituted); and a balance of palm oil as a carrier (suitable oil carrier, excipient, or the like suitable for topical administration may be substituted).

Formulation 8:

A topical formulation comprising 5% by weight squalane; 8% by weight rose hip (Rosa Rubinosa); 7% by weight pomegranate (Rumica Granatum); 5% by weight chia (Salvia Hispanica) seed oil; 5% by weight soybean (Glycinemax, non GMO); 3% by weight lecithin; 8% by weight cholesterol oleyl carbonate (cholesterol annote or cholesterol chloride can be substituted); 3% by weight astaxanthin; 3% by weight tocotriends; 3% by weight lycopene; and 2% by weight alpha d tocopherol; and a balance of palm oil as a carrier (suitable oil carrier, excipient, or the like suitable for topical administration may be substituted).

### EXAMPLE 2

This example is directed to the subtle energy testing of water containing polyester fabric treated with the methods of the invention.

The goal was to measure and determine if there were any energetic differences between a treated fabric sample that is activated according to methods of the invention compared to a control. The treated fabric sample is placed into a copper lined container with electrodes. Copper wire leads are then connected to conductive pads that are imbedded in a ⅛th inch copper container that is 48 inches long by 36 inches wide and 30 inches in height. The articles to be charged, fabric in this Example, are placed inside of the container and a micro current is applied to generate a broad range of frequencies from 2000 hertz (Hz) to 15000 Hz. Here, a micro-current in the frequency of 1300 to 1800 Hz range was applied and controlled by a computer to treat the sample for 5 minutes.

The activated fabric sample and control are then immersed in water in a container to take energy measurments of the activated sample and control fabric. A Electro-Photonic Imager/Gas Discharge Visualization between two samples of Arrowhead spring water was performed by Psy-Tel labs, LLC (Encinitas, Calif.). A Electro-Photonic Imaging (EPI) device, formerly known as a Gas Discharge Visualization (GDV), is an advancement of the original Kirlian photography developed by Dr. Konstantin Korotkov. The technology produces an electric impulse, which generates a response of the object in the form of a electron or photon emission. The glow of the photon radiation due to the gas discharge generated from the electromagnetic field is captured by a digital camera and processed by software that performs statistical analysis of the data to quantify brightness levels (Average Intensity) , size of the glow pattern (Area), Form Coefficient, Entropy, and Spatial Factuality.

The Area parameter gives an indication of the energy of the electrons emitted while the Average Intensity Parameter is proportional to the number of electrons in the sample. A large area indicates that the electrons are easily leaving the sample because they are less tightly bound to the sample. A larger Average Intensity level indicates a larger number of electrons are available in the sample. The parameters of Form Coefficient, Entropy, and Spatial Factuality are related to coherence and a lower value for these parameters suggest better coherence or flow of electrons within the molecules of the sample. This suggest a more homogeneous distribution of the molecules inside the sample.

Results:

The Area was determined for time series of 100 images for each of two water samples. Sample 1 was a control containing water and a polyester fabric sample, and Sample 2 was a sample treated with the energy infusion methods described above. Images were taken and the Area determined and averaged from the 100 image samples, and the results from this parameter show a clear and non-overlapping (statistically significant, with p=0.0136) difference between the charge of the treated and untreated fabric samples. The Average Intensity measurements were also determined and the activated fabric samples showed a higher Average Intensity than the control samples (statistically significant (p=0.00132), data not shown).

User Response

The methods described above were used to make test clothing samples for subject to wear and test. Activated clothing was made from polyester and hemp fiber containing garments. Articles that were pre and post tested consisted of compression sports wear tested on both men and women. Elbow and knee braces, bicycle shorts both polyester blend and specialized copper thread material. Plastic mouth guards, hockey and football helmet inner foam pads, swim wear, athletic shirts, pants and tight fitting shorts, and yoga pants.

Pre and post testing of the polyester and hemp fiber clothing revealed that the person wearing it experienced and demonstrated a increase in specific isolated muscle strength, when manual and computerized measurements were made using a JTECH computerized muscle tester, created by JTECH medical 470 W. Lawndale Dr., Salt Lake City, Utah 84115. Limited testing was also performed using a Cybex computerized muscle tester. Testing results all showed an increase of strength in the individuals ranging from 3% to 22%. Individualized stretch testing was preformed and every individual of a 15 person test showed increased range to motion from 1 to 4 plus inches. Loose fitting clothing and 100% cotton clothing showed no change in the manual or computerized testing. The articles need to be in direct contact with the persons skin (data not shown).

In summary, the results of experiment showed that the fabric sample treated with the above process released more electrons than the control sample. The treated fabric samples also released electrons with a lower average energy, making the water of the treated fabric sample more prone to release electrons that neutralize oxidative stress the underlies many disease processes. Activated garments were made from the methods described herein, and the users that wore the activated clothing items showed an increase in strength and motion in addition to other benefits.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing″, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

11-16. (canceled)

17. An article of clothing manufacture comprising an activated fabric material, wherein said article of clothing manufacture is comprised of a fabric or material activated by the infusion of electromagnetic radiation of a pre-selected frequency or wavelength, duration, and intensity.

18. An article of manufacture of claim 17, wherein at least some of the fabric used to make the article of clothing is made according to a method comprising the steps of:
i) selecting a fabric or other suitable material to use in the manufacture of the article of clothing;
ii) providing a electromagnetic energy frequency generator;
iii) placing said fabric or other suitable material in direct or indirect contact with the frequency generator;
iv) selecting an electromagnetic energy output frequency and level suitable for the particular selected fabric or other suitable material selected in step i); and
v) applying the electromagnetic energy at the output levels selected in step iv) to the fabric or other suitable material from step iii) for a predetermined amount of time;
wherein after step v), the article of clothing or other suitable material comprises an electromagnetic energy infusion charged material that retains this electromagnetic energy charge.

19. An article of manufacture of claim 17, wherein the article of clothing manufacture is selected from the group consisting of: short sleeve shirts, tank top shirts, vest, workout gear, compression sports wear, padded bicycle shorts, elbow sleeves, knee braces, swim wear, wet suits, mouth guards, chin straps, lower and upper back braces, protective ankle braces, and support hose and socks.

20. An article of manufacture of claim 17, wherein said electromagnetic radiation comprises a micro-current of electricity from an electromagnetic energy frequency generator where the generated current is in direct contact with a copper container or cage in which the fabric or other suitable selected material is placed.

21. An article of manufacture of claim 17, wherein the article of clothing manufacture is selected from the group consisting of tee shirts, sports compression clothing, pants and shorts for yoga, wet suits, support socks and hosiery, bicycle shorts and similar close fitting work out shorts, footwear, and swim wear.

22. An article of manufacture of claim 17, wherein said fabric or material activated by the infusion of electromagnetic radiation is activated by electromagnetic energy applied by a frequency generator.

23. An article of manufacture of claim 17, wherein said fabric or material activated by the infusion of electromagnetic radiation is activated by a micro-current of electricity and said electromagnetic radiation consist essentially of a micro-current of electricity.

24. An article of manufacture of claim 17, wherein said fabric or material activated by the infusion of electromagnetic radiation is activated by sound waves and said electromagnetic radiation consist essentially of sound waves.

25. An article of manufacture of claim 17, wherein said fabric or material is activated by the infusion of electromagnetic radiation that utilizes a self-sustaining frequency.

26. An article of manufacture of claim 17, wherein said fabric or material activated by the infusion of electromagnetic radiation is applied by a frequency generator that is placed in direct physical contact with the fabric or material.

27. An article of manufacture of claim 17, wherein said fabric or material activated by the infusion of electromagnetic radiation is applied by a frequency generator that is placed in direct physical contact with a copper container or cage in which the fabric or other material is placed.

28. An article of manufacture of claim 17, wherein said fabric or material activated by the infusion of electromagnetic radiation is applied by a frequency generator that is placed in indirect physical contact with the fabric or other material.

29. An article of manufacture of claim 17, wherein said fabric or material activated by the infusion of electromagnetic radiation is applied by a frequency generator in direct physical contact with a fluid in which the fabric or other material is placed or bathed.

30. An article of manufacture of claim 17, wherein said fabric or material activated by the infusion of electromagnetic radiation is applied by a frequency generator in direct physical contact with the fabric or material selected.

31. An article of manufacture of claim 30, wherein said frequency generator comprises electrodes that are in direct contact with said selected material.

32. An article of manufacture of claim 17, wherein said fabric or material activated by the infusion of electromagnetic radiation is activated by electromagnetic energy applied by a frequency generator.

33. An article of manufacture of claim 17, wherein said fabric or material activated by the infusion of electromagnetic radiation is activated by a fluctuating electromagnetic (EM) field generated by a radio (RF) oscillator.

34. An article of manufacture of claim 17, wherein said fabric or material activated by the infusion of electromagnetic radiation is activated by an infrared (IR) light generating source in the infrared spectral range into the selected material.
